(19) United States
(12) Patent Application Publication
(10) Pub. No.: US 2013/0237797 A1
(43) Pub. Date: Sep. 12, 2013

(54) DEVICE AND METHOD FOR DETERMINING A BIOLOGICAL, CHEMICAL AND/OR PHYSICAL PARAMETER IN A LIVING BIOLOGICAL TISSUE

(71) Applicants: Arno Müller, Ulm (DE); Heinz-Peter Utz, Berlin (DE)

(72) Inventors: Arno Müller, Ulm (DE); Heinz-Peter Utz, Berlin (DE)

(73) Assignee: Vivantum GmbH, Berlin (DE)

(21) Appl. No.: 13/639,574

(22) PCT Filed: Mar. 31, 2011
(86) PCT No.: PCT/EP2011/054977

§ 371 Date: Apr. 25, 2013

(30) Foreign Application Priority Data
Apr. 13, 2010 (DE) 10 2010 014 775.3

(51) Int. Cl. A61B 5/00; A61B 5/145

(52) U.S. Cl. CPC A61B 5/0059; A61B 5/14532
USPC 600/407

(57) ABSTRACT

The invention relates to a device for determining biological, chemical and/or physical parameters in living biological tissue, comprising an energy supply unit, a laser operating unit with at least one laser source directed at the biological tissue, at least one sensor unit for detecting the light scattered back and/or absorbed by the biological tissue, a control unit, a storing and processing unit and an interface for an external data processing unit. The method according to the invention includes execution of a calibrating phase for ascertaining a reference set (R) of reference vectors (Ri), in each case involving independently ascertaining a parameter (BZi), radiating unpolarized laser light onto the biological tissue and registering a measured value vector (Mi) from a series of optical measured variables and executing an interpolation phase for ascertaining an interpolation set (I) of interpolation vectors (Ik), in each case involving radiating unpolarized laser light onto the biological tissue and registering a measured value vector (Mk) from a back-scattered light intensity with a subsequent determination of an interpolated parameter (BKk) from the reference set (R).

The invention relates to a device according to claim 1 for determining biological, chemical and/or physical parameters in a living biological tissue, and a method according to claim 15 for determining biological, chemical and/or physical parameters in a living biological tissue.

Determining biological, chemical and/or physical parameters in a living biological tissue is a fundamental necessity in the field of physiological research and in medical examination processes. A particular example is in this case the identifying and monitoring of blood components, and especially the determining of blood glucose concentration. Usually, tissue needs to be injured for this purpose and a certain amount of blood taken. Although devices are available nowadays for such invasive processes by means of which taking blood is possible at minimum expenditure and in a relatively safe manner, some individuals perceive this as being unpleasant. In addition, taking blood always has to be associated with particular precautions for persons having blood coagulation disorders in order to avoid unstoppable bleeding and hence severe complications. A time continuous control of blood glucose and other blood parameters is hardly possible for such persons or only under a physician's guidance and survey.

So as to overcome the cited problems, methods have been developed by means of which blood glucose concentration can be determined non-invasively, i.e. without puncturing and withdrawing blood. Such methods rely on measuring light absorption or a change of the polarization state of a light irradiated on the tissue.

US patent document U.S. Pat. No. 5,383,452, for instance, discloses a method in which the polarization plane rotation caused by the sugar concentration in biological tissue is measured. By means of calibration performed in advance using conventional blood glucose measurement methods in conjunction with deliberately influencing the blood glucose level within the framework of a tolerance test, the rotation of the polarization plane can be used as a measure for the blood glucose concentration.

German published patent application DE 43 14 835 A1 discloses a method and a device for analyzing glucose in a biological matrix, in which light is injected at a location into the matrix, and the intensity of the light measured within the matrix is determined. The measure intensity is then used as a measure for the glucose concentration within the matrix.

The non-invasive determination of the blood glucose level thus is comparably simple due to the physically known interaction between light and glucose. Determining physical values in living tissue respectively ascertaining laboratory values in human blood, however, is not limited exclusively to the determination of the glucose level but encompasses a much larger amount of values to be measured. The non-invasive methods known from the prior art are no longer sufficient for this purpose. In particular the knowledge about the polarization state or the intensity of the scattered light is not sufficient to non-invasively ascertain the parameters in question. The measurement methods mentioned at the beginning thus reach their limits.

Consequently, the object is to provide a non-invasive method and a device for realizing the method by means of which biological, chemical and physical parameters can be determined in living tissue even under unfavorable or unknown or physically not yet sufficiently precisely researched interactions between the light on the one hand, and the parameter to be measured on the other.

The object is achieved by means of a device according to claim 1 and a method according to claim 13. The respective dependent claims contain appropriate and/or advantageous embodiments of the device and method.

The device according to the invention for determining biological, chemical and/or physical parameters in living biological tissue includes an energy supply unit, a laser operating unit comprising at least one laser source directed onto the biological tissue, at least one sensor unit for detecting light scattered back and/or absorbed by the biological tissue, a control unit, a memory and processing unit, and an interface for an external data processing unit.

The sensor unit is appropriately realized as a planar sensor array. The first sensor portion forms an inner sub-array, and the second sensor portion an outer sub-array surrounding the inner sub-array. The distribution of the scattered light can thereby be detected depending on location.

In an appropriate configuration, the inner sub-array comprises an attachment having a polarizer oriented in a first polarization direction, and the outer sub-array comprises an attachment having a second polarizer oriented in a second polarization direction, wherein the first polarization direction is oriented perpendicular to the second polarization direction. The scattered light can thus be detected depending on location and direction on the one hand, and in its polarization state on the other.

In a further embodiment, the sensor unit is realized as a photometer unit having a first photometer for determining an absolute intensity of the light from the laser source, and a second photometer for measuring the light scattered by the tissue. The sensor unit comprises in an appropriate configuration a change-over mechanism for redirecting the light from the laser source to the first photometer as required.

In an appropriate embodiment, two laser sources having mutually orthogonal beam directions are provided. This allows characteristics of the scattered light to be detected depending on the beam direction of the incident light.

The laser source is appropriately arranged in a hole situated on the sensor array and has a beam direction inclined at a tilt angle with respect to the detection direction of the sensor array. It is advantageous for the tilt angle to have a value adjustable to about 45°. Thereby, the scattered light generated at a certain depth within the tissue rather than the light reflected on the tissue surface is detected by the detector arrangement.

Appropriately, the first sub-array consists of at least one first single diode, and the second sub-array of at least four single diodes which are uniformly distributed around the first single diode.

In an appropriate embodiment, the sensor unit comprises a pressure sensor for measuring the contact pressure between the sensor unit and the tissue, and/or a temperature sensor for measuring tissue temperature. This allows the contact pressure of the sensor unit on the tissue to be monitored on the one hand, and the dependence on the contact pressure of the parameters to be measured on the other. The temperature sensor serves likewise to monitor constant measuring conditions.

Appropriately, the pressure sensor and/or temperature sensor form(s) a control circuit cooperating with the control unit for setting an appropriate contact pressure and/or an appropriate temperature value.

The method according to the invention for determining a biological, chemical and/or physical parameter in a living biological tissue is realized in the form of a self-learning process flow including the following process steps:

The process is divided into two basic process blocks, this being a calibrating phase on the one hand, and an interpolation phase on the other.

Realizing the calibrating phase comprises at least one conventional determination of the parameter in conjunction with at least one light scatter measurement performed on the tissue for determining optical measured values. In connection therewith, the at least one conventionally determined parameter is assigned to the respective optical measured values. These data pieces are stored as a calibrating reference set.

Realizing the interpolation phase comprises at least one light scatter measurement performed on the tissue for determining optical measured values. The parameter to be determined is interpolated from the measured values of the light scatter measurement and the data of the reference set. The interpolated parameter is stored in the reference set.

When the calibrating phase is realized, the determining of a reference set is appropriately realized in the form of reference vectors. Each reference vector consists of the conventionally determined parameter and a measured value vector including the optical measured values. When the interpolation phase is realized, a measured value vector containing optical measured values is determined and the associated interpolated parameter together with the measured value vector is transferred into the reference set as a new reference vector.

The measured value vector ascertained when realizing the calibrating phase includes in an appropriate embodiment a light intensity influenced by the tissue in a first polarization direction, and a light intensity influenced by the tissue in a second polarization direction. The measured value vector is combined with the independently ascertained parameter to result in the reference vector.

The measured value vector ascertained when realizing the interpolation phase includes in an appropriate embodiment a light intensity influenced by the tissue in a first polarization direction, and a light intensity influenced by the tissue in a second polarization direction.

The interpolated parameter is ascertained using the following steps:

The measured value vector is registered and the closest measured value vectors are determined from the reference set having a minimum distance to the measured value vector. Subsequently, the parameter assigned to the registered measured value vector is interpolated from the closest measured value vectors and the respectively associated reference parameters.

The interpolated parameter is added to the reference set together with the measured value vector after realizing the interpolation.

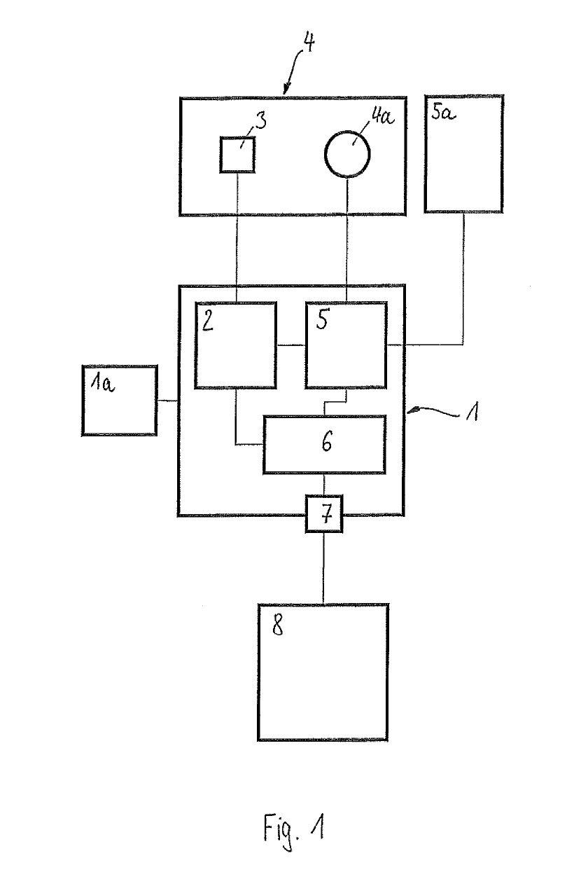

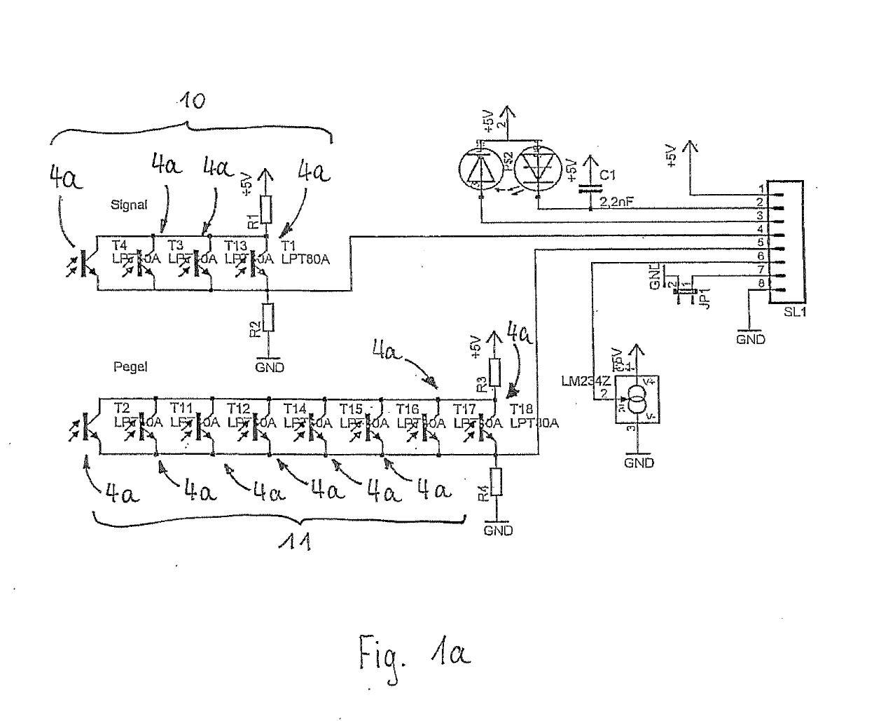

The device and the method according to the invention will be explained hereinafter in more detail on the basis of exemplary embodiments. FIGS. 1 to 15 serve the purpose of clarification. The same reference numerals are used for identical parts and method steps and/or parts and method steps of equal action.

In the Figures:

FIG. 1 shows an exemplary block diagram of a device according to the invention,

FIG. 1a shows an exemplary circuit diagram of a plurality of measuring sensors,

FIG. 1b shows an exemplary circuit diagram of a central unit,

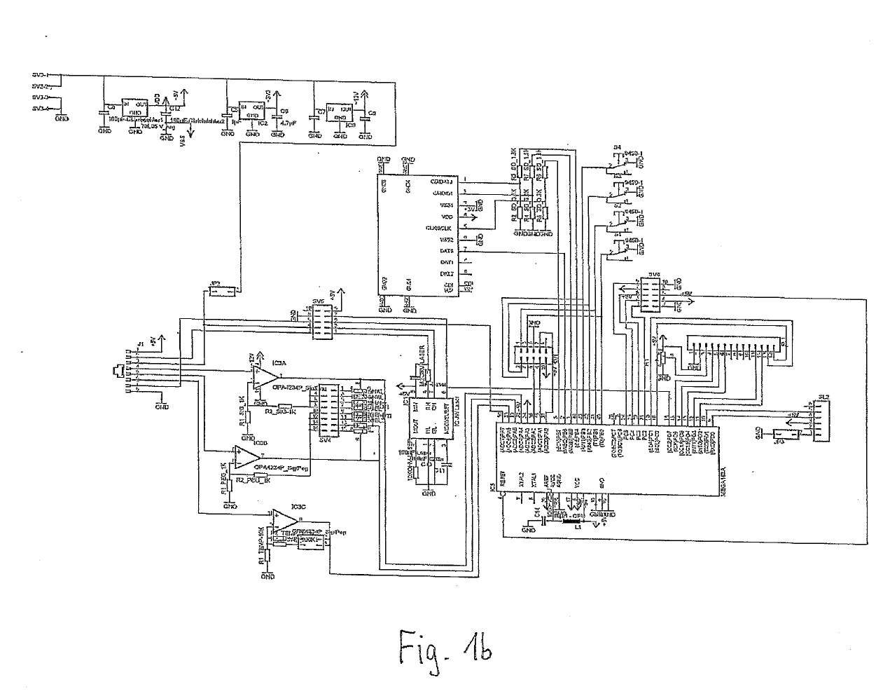

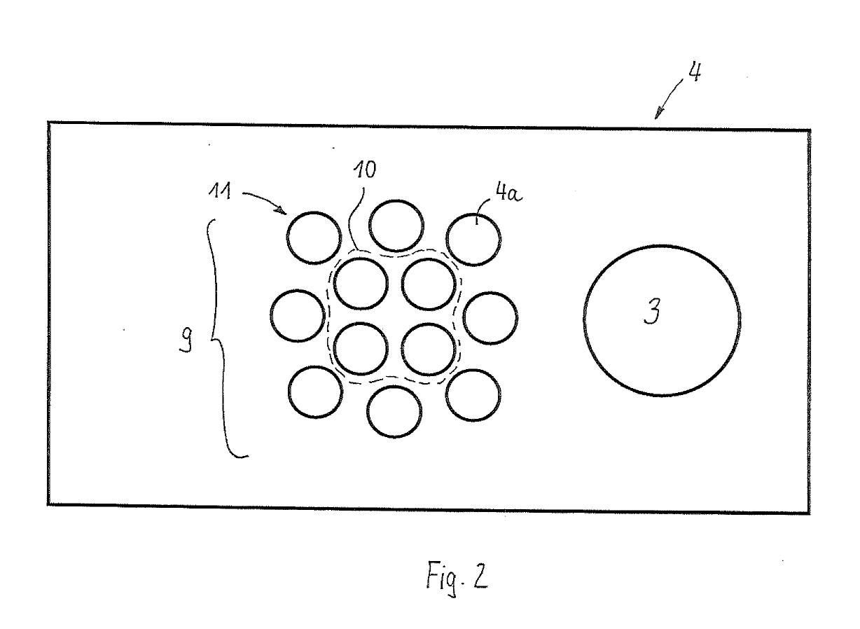

FIG. 2 shows an exemplary representation of a sensor unit,

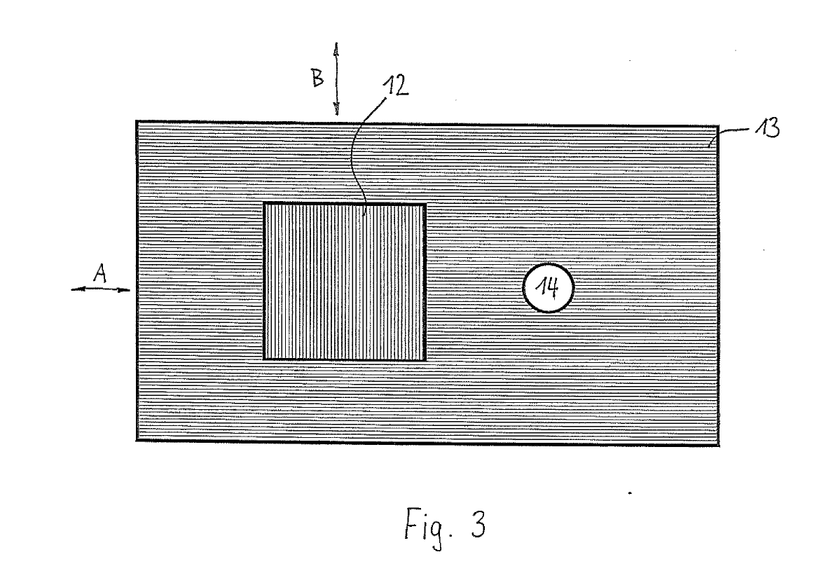

FIG. 3 shows a covering of the sensor unit shown in FIG. 2 with polarizers,

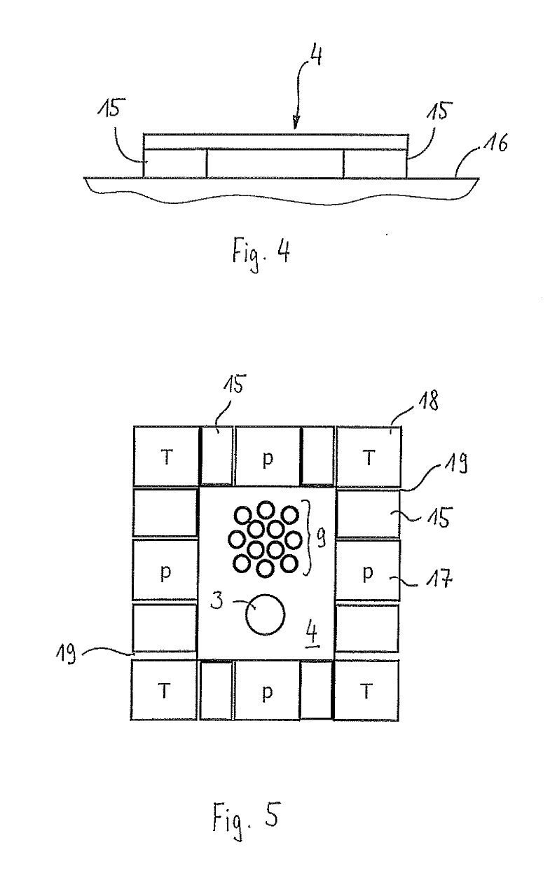

FIG. 4 shows a sensor unit completed with further components in a side elevation in a sectional view,

FIG. 5 shows a sensor unit completed by spacers and pressure and temperature sensors,

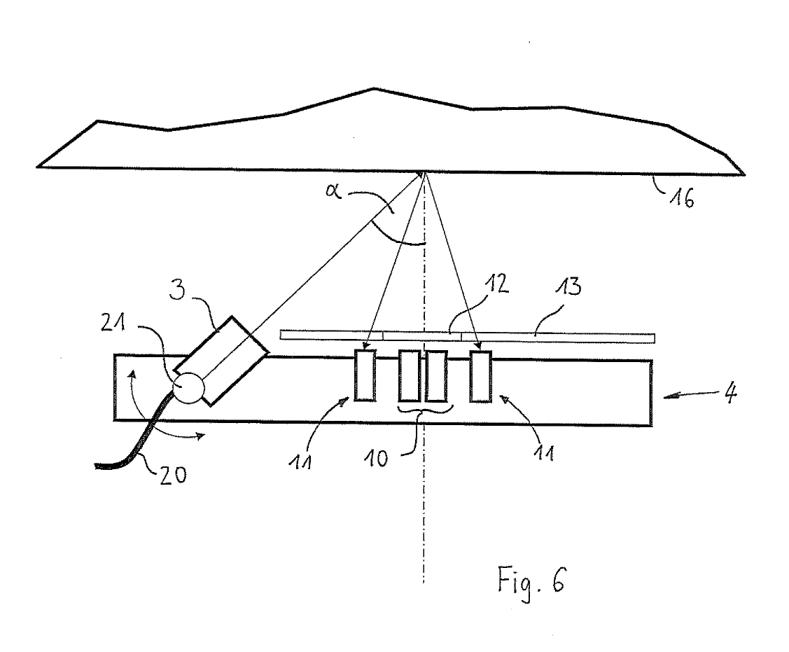

FIG. 6 shows the optical path provided for the sensor unit in a first exemplary embodiment,

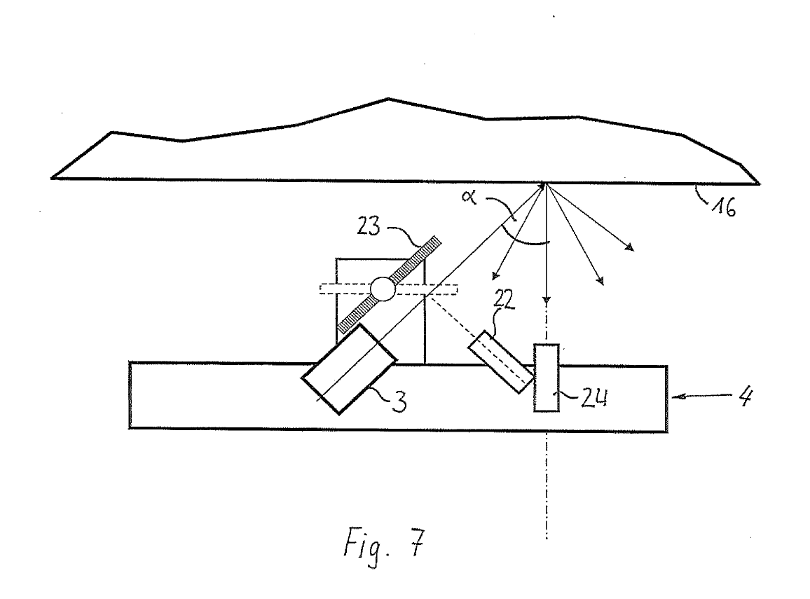

FIG. 7 shows an embodiment of a sensor unit for an optional absolute measurement of the initially emitted laser intensity,

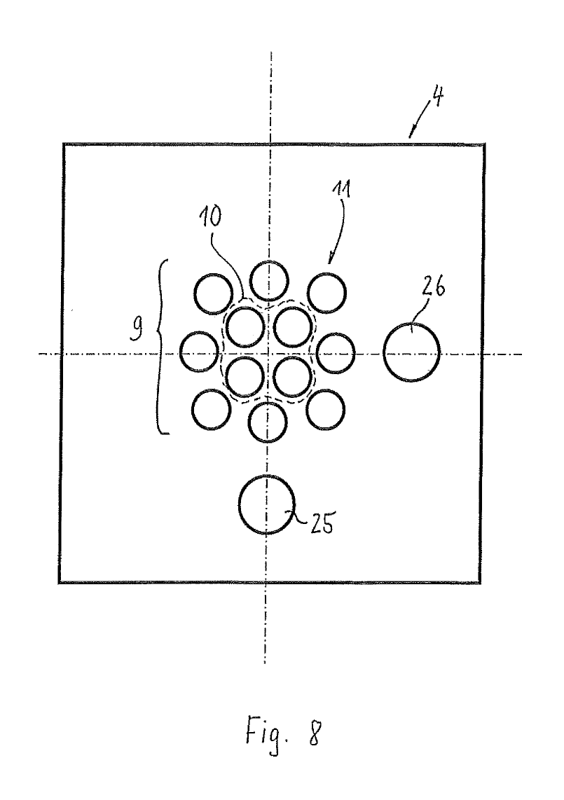

FIG. 8 shows an embodiment of a sensor unit having two laser light sources with mutually orthogonal beam directions,

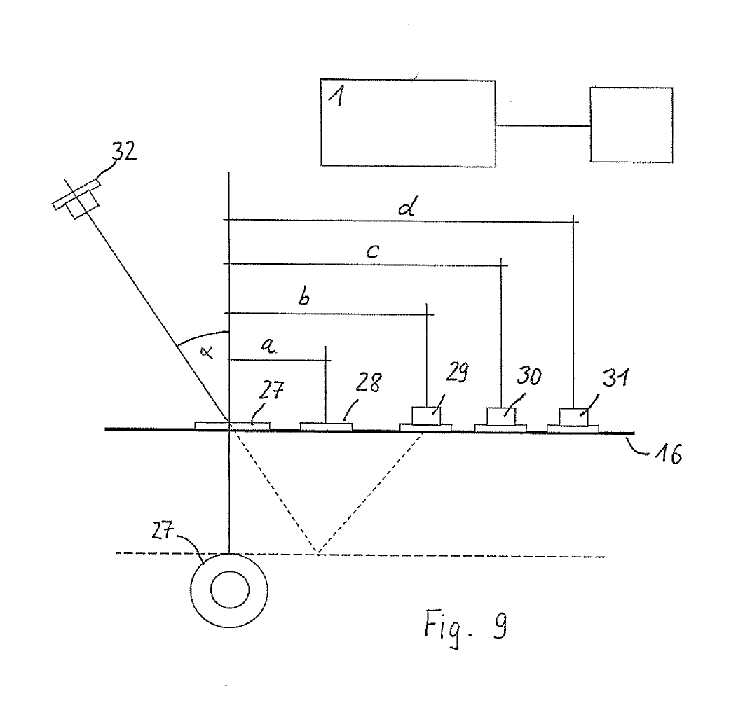

FIG. 9 shows a further exemplary sensor arrangement,

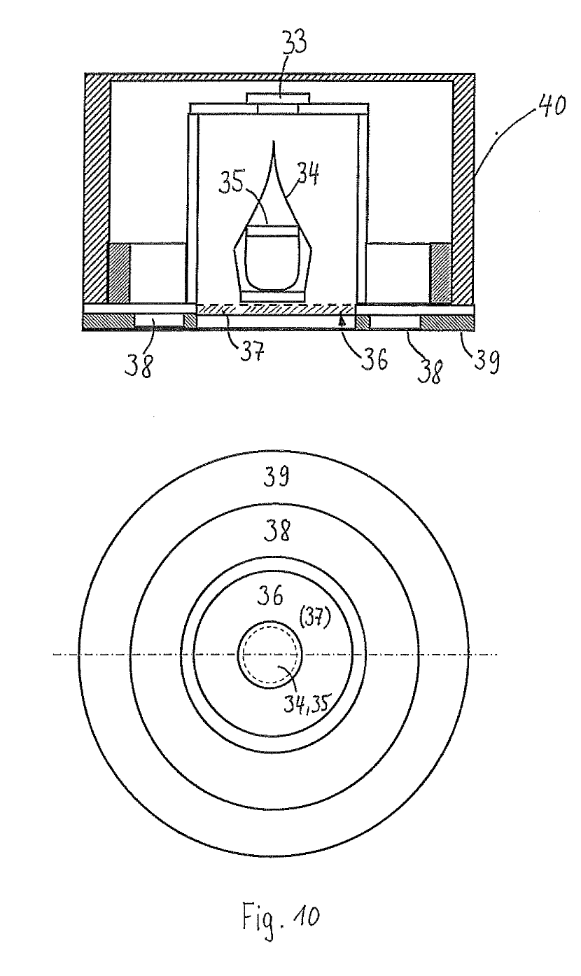

FIG. 10 shows a further embodiment of a combined arrangement of sensor and light source,

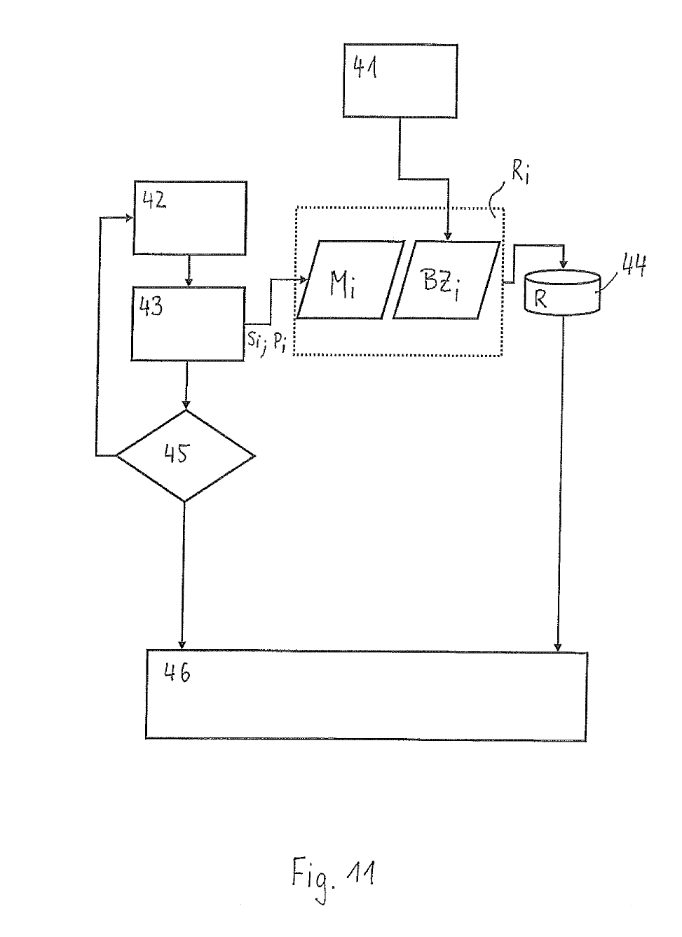

FIG. 11 shows an exemplary representation of a calibrating phase flow chart,

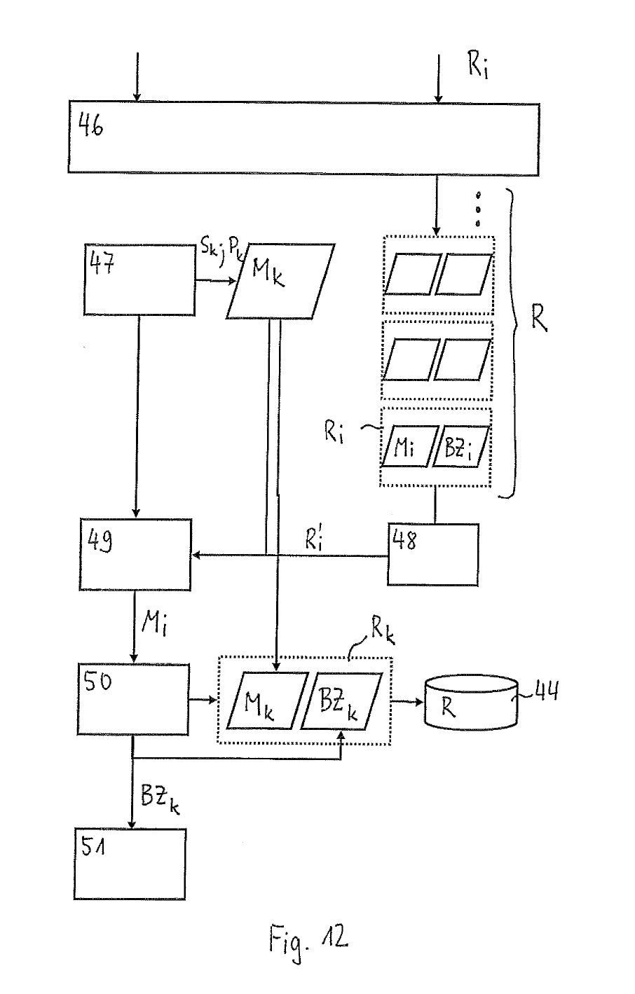

FIG. 12 shows an exemplary representation of an interpolation phase flow chart,

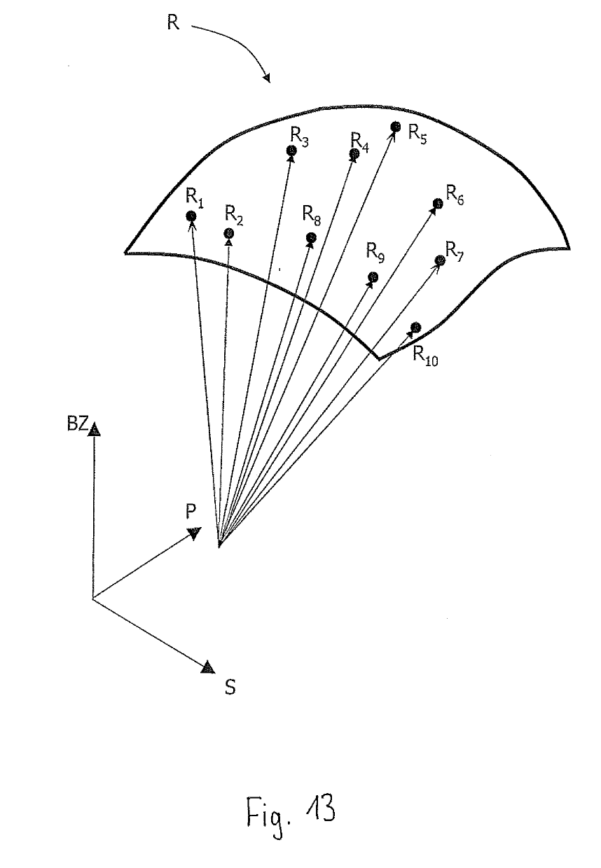

FIG. 13 shows a schematic reference set,

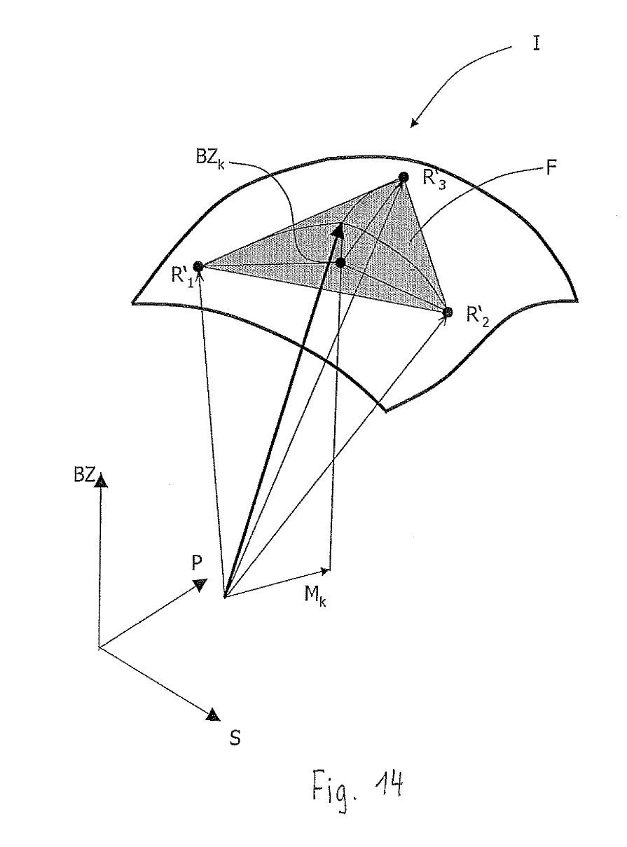

FIG. 14 shows an interpolation realized on the reference set,

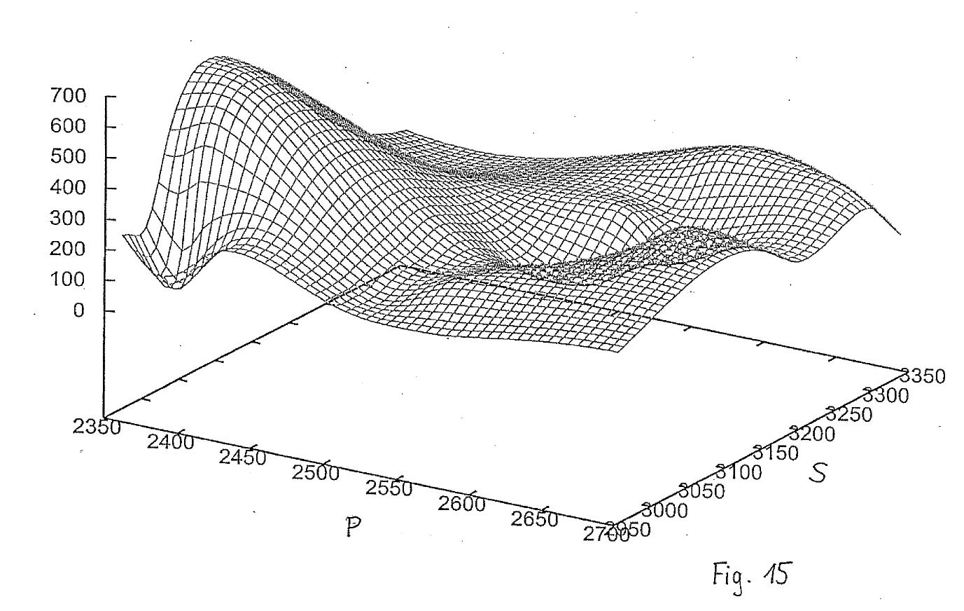

FIG. 15 shows a reference set ascertained from real measurements.

FIG. 1 shows an exemplary block diagram of a device according to the invention, FIG. 1a in connection therewith an exemplary circuit diagram of measuring sensors, and FIG. 1b an exemplary circuit diagram for realizing a central unit by means of integrated circuits. Use is made of a modular concept in building up the device. This modular concept allows various components, sensors, data processing units and further equipment to be combined such that an amount of measured data as extensive as possible and adapted to the single case can be detected and processed.

The device consists of a central unit 1 which is powered via an energy supply unit 1a. As the energy supply unit, a mains connection having a downstream transformer and rectifier circuit as well as an accumulator or battery unit can be used.

Within the central unit, a laser operating unit 2 is provided. Same controls a laser source 3 which can be connected to the central unit, or contains itself a laser device from which the laser light is guided to the outside via a fiber-optic light cable. In such a case, the laser source 3 is merely a beam optics downstream of the fiber-optic cable for aligning the beam toward the tissue surface.

As the laser operating unit, the usual driver hardware for this purpose can be employed. Same appropriately allows the laser source to be operated in a pulse mode with variably adjustable time intervals in the range of from 100 ms to 800 ms, and hence supports pulse programs to be executed.

As the laser source, a laser diode having an emitted wavelength of between 800 nm and 950 nm is appropriately used. The power of the laser diode should appropriately be limited to a few mW so as to avoid damages within the tissue. It is possible to use a P type laser diode. Appropriately, the laser diode is protected against surge voltages by a capacitor circuit.

For obtaining measured values, at least one sensor unit 4 is provided. Same includes at least one measuring sensor 4a which receives the laser light scattered, reflected, attenuated or otherwise influenced by the biological tissue. In the present example, at least the emitting opening of the laser source 3 is integrated together with the measuring sensor 4a into the body of the sensor unit 4. The sensor unit 4 in the present example hence forms a measuring module connected to the central unit 1 for emitting laser radiation and obtaining measurement data.

The usual photo diodes for this purpose can be used as the measuring sensors. Photo diodes having a light receiving diameter of about 2 to 5 mm have turned out to be appropriate in this case. In identifying scattered radiation in the infrared spectral range, a black covering of the light receiving surface is appropriate so as to preclude the diode being influenced when visible light is incident. In order to achieve a higher sensitivity of the sensor unit arrangement and to detect a sufficiently large measuring area, it is appropriate to combine and suitably interconnect, in particular in parallel, some photo diodes in sets and sub-arrays 10 and 11. An example for this is shown in FIG. 1a. The sensitivities of the photo diodes may in this case be adjusted by corresponding resistors R1, R2, R3 and R4 which are integrated into the circuit in appropriate locations. The circuit necessary for this and the arrangement of the photo diodes on the respective circuit board form an integral part of the sensor unit.

For operating the sensor unit 4, in particular for receiving the measurement signals detected by the measuring sensor, a control unit 5 is provided within the central unit. Same cooperates with the laser operating unit 2. The control unit supplies switching signals to the laser operating unit and includes at the same time an amplifier for the measurement signals collected by the sensor unit and the pressure and temperature sensors.

A standard amplifier circuit can be used for amplifying in which the gain factor can be very easily adjusted by a ratio of resistors employed in this case. Various gain factors can be used in this case for different sensor groups. For example, a gain factor of 10 is possible in converting measurement signals of the temperature sensor, and a gain factor of 1 in converting the measurement signals from the measuring sensors of the sensor unit. These different gain factors may be usually predefined via setting jumpers on the circuit board of the amplifier circuit.

Both components are applied with control signals from a storing and processing unit 6 and implement in this case a measuring program stored in the storing and processing unit. Optionally, additional sensors 5a can be connected to the control unit 5. Same can in particular be pressure or temperature sensors.

Using a temperature sensor is appropriate to monitor a constant temperature in the tissue to be measured and thus to prevent the measuring process to be negatively influenced. Temperature sensors usual for such measurements can be used for this purpose.

The connection between the single components, for instance, is realized by an eight-core cable, in particular a network cable.

The physical effects and interactions of light in the biological tissue detected by the optical measuring sensors can be of quite different nature. However, they will be known as such to the person skilled in the art, although the precise consequences of each single effect for the measurement signals finally detected by the sensor arrangement can be very complex in their entirety. As the fundamental effects have to be mentioned in this point the light absorption within the tissue according to the law of Lambert/Beer, the diffraction of light at the interface of various dielectric materials, in particular the tissue surface and air, which can be described physically by means of the Fresnel equations. A diffraction or light scattering occurring within the tissue, which can be both direction-dependent and diffuse and can in particular be described as a Rayleigh or Mie scattering and depends on the size of the scattering particles, as well as mainly polarization effects, in particular rotations of polarization planes and other forms of optical activity especially caused by chiral centers of molecules present within the tissue can likewise be exploited as physical interaction processes for obtaining measured values.

The storing and processing unit 6 can be programmed for this purpose, the data and measured values stored in same can be read out and processed externally or else be changed. For this, an interface 7 is provided via which an external data processing unit 8, e.g. a computer or an external network can be connected. The central unit acts in this case as a data collecting means which can be consulted regularly. This may be performed in particular via a USB interface.

As an alternative, the interface may also be implemented in the form of an SD card. Same can be inserted as a mobile memory module into a corresponding slot of the device and loaded with the measured data. Said pieces of data are subsequently read out in a computer.

The components, of course, can all be accommodated in a housing and miniaturized. It is easily possible for the arrangement to be realized as a device portable on a part of the body, e.g. a bracelet. The elements present in the central unit are in this case sufficiently miniaturized and appropriately even arranged on a circuit board of the sensor unit 4.

The use of a hardware architecture using a microcontroller is in this case appropriate. Same in particular executes an AD conversion at a processing width of 10 or 12 bits. When an AD converter having a processing width of 10 bits, and an analogous input signal having a maximum voltage of about 4000 mW is used, a resolution of about 3.9 mV/unit is achieved in this case. At this point, it is advantageous to reserve a voltage range as large as possible for the input signal since the level of the actually applied measurement signals is not known a priori. An overflow of the AD converter is in this case avoided. However, the resolution of the AD conversion is reduced in this case.

An EEPROM for buffering process data is advantageous. As the clock frequency, a frequency interval of between 1 MHz and 8 MHz and more can be used depending on the specific configuration of the microcontroller. The microcontroller exhibits a series of ports via which the measurement signals of the sensor unit and further sensors can be read in, and via which a programming of the microcontroller can be performed. Programming is in particular performed via an integrated JTAG circuit. Moreover, ports for storing the measured data in particular in an SD card and the transfer thereof to an external data processing unit are provided. A port ultimately serves to output control signals to the control unit and the laser operating unit for activating and deactivating the laser source and/or sensor unit and other measuring sensors.

A further appropriate device not shown here can be a means for a wireless data transmission which is arranged in the central unit and by means of which it is possible to send the ascertained measured data to an external receiver, e.g. a medical equipment or central surveillance unit. The entire device can in this case have the outer shape of a mobile telephone.

The entire device appropriately comprises a display not shown here. As the display, small and simple liquid crystal displays for miniaturized devices as well as bigger displays for configurations that can be stationarily employed can be used. The display can be realized as a standard hardware in combination with a corresponding driver library.

A series of keys is provided for user guidance. In conjunction with an interface for user guidance, same allow device parameters to be set and cancelled, for storing and reading out measured data and such similar data. Four keys are provided in a minimum configuration. Same access the microcontroller via corresponding ports. When a key is pressed, the corresponding port is connected to ground and thus the digital input generated. An internal program code for reading the keys counts the applied bytes and checks same for changes. In accordance with the results obtained in this case, corresponding menus on the display are activated, deactivated or scrolling functions executed within the menus.

FIG. 2 shows a principle representation of the surface of an exemplary sensor unit 4 directed towards the tissue to be examined. In the example shown here, the sensor unit includes a planar sensor array 9 composed of single measuring sensors 4a. The number of measuring sensors is basically arbitrary. In the present example, the sensor array 9 is subdivided into a first sensor portion having an inner sub-array 10 of four measuring sensors, and a second sensor portion having an outer sub-array 11 of eight measuring sensors. The outer sub-array encloses in this case the inner sub-array completely. As already mentioned before, the laser source 3 or a corresponding beam optics is embedded into the sensor unit body next to the sensor array. In terms of measurement engineering, it is also possible for single measuring sensors to be excluded from each of the two sub-arrays or to be combined arbitrarily. This allows various configurations of the sub-arrays to be realized. It is in particular possible for the measuring sensors being closest to the laser source to be switched off or to weight their signals less in terms of measurement engineering than those of the remaining measuring sensors.

As shown in FIG. 3, the inner sub-array 10 is in this case covered by a first polarizer 12, and the outer sub-array 11 by a second polarizer 13. Same have mutually orthogonal polarization directions A and B. The light emitted by the laser source 3 is not influenced by the polarizing cover. For this reason, the polarizer 13 has an opening 14 through which the laser light can pass. The diameter of the opening can be in a range of from 1 to 3 mm. As an alternative, of course, arranging a shutter having a variable opening cross-section is also possible here. For covering the sub-arrays or the surface of the sensor unit, use is appropriately made of polarization foils which are fastened on a glass substrate and thus form a planar cover on the sensor surface.

The construction of the sensor unit shown in FIGS. 2 and 3 can be completed by further components. FIG. 4 shows an embodiment in this respect in a side elevation, and FIG. 5 in a view of the sensor area. The additionally added components are intended to ensure sufficient spacing between the sensor area and the surface of the tissue on the one hand, and to detect parameters on the other which are necessary for a smooth measuring process.

In the example shown in FIG. 4 and FIG. 5, spacers 15 evenly distributed around the sensor surface are provided between the sensor area and the tissue. The spacers touch down on the tissue surface 16. If need be, they have an adhesive contact surface which prevents the entire arrangement from slipping and fixes the sensor in the allocated place on the tissue.

The spacers are situated within an arrangement of pressure sensors 17 and temperature sensors 18 surrounding the sensor area. The pressure sensors 17 register the contact pressure of the pressure unit on the tissue surface and are coupled to the control unit explained above within the central unit. The temperature sensors register the temperature directly on the tissue surface on the one hand, and in the direct outer environment of the measuring site on the other. They have a contact surface which ensures good thermal contact between the tissue surface and the sensor body.

The spacers 15 and the intermediately arranged pressure and temperature sensors 17 and 18 are separated from one another by air-permeable slots 19. These slots prevent a measured value-distorting negative pressure between the sensor area and the tissue surface and a consequential increased blood circulation or another kind of distorting change of the tissue.

FIG. 6 shows an exemplary optical path on the sensor unit 4 described above. The laser light emitted by the laser source 3, if need be introduced by a fiber-optic light cable 20, impinges under a finite angle α within a beam spot of a finite size onto the tissue surface 16 and penetrates there into the uppermost tissue layers. The scattered light generated within the tissue propagates from the beam spot within a scattering cone and is detected in a detection direction oriented perpendicular to the tissue surface. In this process, the scattered light penetrates the polarizers 12 and 13 and is received by the sub-array 10 and 11 arranged behind. The angle of incidence a is about 45° and can be adjusted around this angle by means of a tilting mechanism 21 arranged in the sensor unit. Using such a sensor unit allows for the intensities at both sub-arrays to be determined in a relative measurement.

FIG. 7 shows a further development of the arrangement shown in FIG. 6, in which, apart from the relative measurement of the intensities impinging on the two sub-arrays, an absolute measurement of the intensity of the laser light initially emitted to the tissue surface is possible. Two sets of measuring sensors are provided for this purpose. At least one of the measuring sensors is in this case intended exclusively for the absolute intensity measurement. In the example shown here, this is a measuring sensor 22. Its detection direction is directed against the surface of a deflecting mirror 23, which can be optionally pivoted into the radiation direction of the laser source 3 and thus deflects the emitted laser light directly to the measuring sensor 22. The change-over mechanism for the deflecting mirror is likewise addressed by the central unit, in particular the control unit included in same.

The sensor arrangement shown in FIG. 7 furthermore includes the usual measuring sensors which are sensitive to the light scattered from the tissue surface. In the example shown in FIG. 7, a single measuring sensor 24 is shown for this purpose by way of example. Instead of this single measuring sensor, of course, the sub-arrays 10 and 11 shown in the preceding figures can likewise be provided. One of the measuring sensors of the outer sub-array 11 can in this case be utilized in line with the present exemplary embodiment as the measuring sensor 22, and is correspondingly tilted down.

FIG. 8 shows a further sensor area having two laser light sources 25 and 26 in combination with the array arrangement 9 of the sub-arrays 10 and 11 already described above. The laser light sources 25 and 26 have beam directions of mutually orthogonal orientation and are inclined at an angle of 45° with respect to the tissue surface. The array arrangement 9 thus registers scattered light that has been generated in the tissue by the laser light source 25 on the one hand, and scattered light is detected by the same array arrangement on the other that is caused in the tissue by the laser light source 26.

The device shown in FIG. 8 is appropriately operated in a pulse mode. At this point, the laser light source 25 is firstly activated by the laser operating unit 2 within the central unit, while the array arrangement in turn detects the light scattered from the tissue. The laser operating unit 2 activates next the laser light source 26, and the measuring procedure in the array arrangement is repeated so that four measured values are obtained in total within this measuring cycle.

FIG. 9 shows a further example of a sensor arrangement. Same consists of an arrangement which is applied to the tissue surface 16 and consists of an annular detector 27, a photo detector 28 for absorption measurement, a photo detector 29 for refraction measurement, a photo sensor 30 having a spectral resolution for determining wavelength-dependent absorption, and a photo sensor 31 for determining the polarization state of the light scattered in the tissue. A laser source 32 having an irradiation angle α of about 45° serves as the light source. The central unit 1 already mentioned controls the operation of the laser source and sensor arrangement.

The annular detector 27 receives the scattered light generated in the tissue and, if need be, is laterally screened off against possibly incident undesired light fractions. For the position and the operation of the photo detector 28 and the photo detector 29, the average light path to be covered within the tissue needs to be taken into account. The distances a to d within the arrangement have to be chosen such that an optimum of the signals arising at each detector is achieved. The penetration depth of the irradiated laser light resulting within the tissue can be varied by the power and wavelength of the light. Since the penetration depth of light in biological tissues changes with the wavelength, the distances a to d consequently need to be changed accordingly.

Apart from the angle of incidence of 45°, other angles or even a grazing incidence is/are possible. The spectral detection of the scattered light at the photo detector 30 allows a chemical analysis of the examined tissue.

FIG. 10 shows a further embodiment of a combined sensor and light source arrangement. The arrangement consists of a housing with an arrangement of a light source 33, in particular a laser source, and optically reflecting surfaces 34 and 35 contained therein. Same reflect the laser light a multiple of times and cause it to exit from an opening 36 situated on the underside of the arrangement. The opening is spanned by a polarization foil 37. Concentrically arranged annular detectors 38 and 39 are disposed around the opening 36, while the entire arrangement is housed in a housing 40 having a preferably black lacquer coating.

For measuring diffraction effects, the annular detectors, for instance, contain photo layers and/or solar layers and can also be realized as a unit. The non-linearity between the measuring signal and the irradiated light intensity possibly existing in the detectors can be balanced by varying the irradiation power. Pressure and/or temperature sensors can be present. One or more of the sensors from the FIG. 10 exemplary embodiment or else from the previously shown exemplary embodiments can also be used as reference detectors which detect and correct errors when the sensor arrangement repeatedly touches down.

The laser sources mentioned above radiate appropriately in a wavelength range in which the penetration depth of light into the tissue is maximum. Laser sources are useful for this purpose, the emitted light of which has a wavelength of about 650 nm to 1000 nm and is therefore in the near infrared. Light of such a wavelength, for example, penetrates into human skin up to a depth of 4 cm and reaches an intensity there which amounts to 25% of the initial value. Laser diodes in the red and infrared spectral range, in particular semiconductor lasers or color center lasers have stood the test in this case. At this point, relatively short laser pulses of about 200 ms are sufficient.

Of course, it is also possible to use other wavelengths of the electromagnetic spectrum to obtain sensitive statements on various tissue layers. Thus it is possible, for instance, to irradiate light in the UV range at a wavelength of less than 400 nm and thus to reach penetration depths of up to 1 cm in order to only examine dermal tissue layers selectively.

The wavelength of the light used, however, also depends on the tissue liquids present in the examined tissue. When a tissue of a high blood supply, e.g. mucous membranes, is examined, or a vein portion is directly measured, the wavelength of the light should be selected such that the oxygen saturation given in the blood is not an issue.

In particular body cavities are possible as preferred locations of the measuring method. Thus it is possible to perform a measurement in the umbilicus area.

The precise parameters for configuring a measurement program can in this case be entered into and adjusted in the central unit via input means present in same, in particular buttons, touch screens, but also via an external interface. The first embodiment is in particular suited for larger, stationary installations, the latter option making sense for small mobile devices and miniaturized measuring arrangements.

In this context, it is advantageous to provide means for user guidance to the user of the measuring arrangement which are realized, for instance, in the form of signal tones, voice outputs, displayed font or symbol representations, menu sequences and similar further signaling means. This concerns both the execution of configurations at the central unit and the execution of measurements or else the management of user data and measurement series.

The measurements as such should preferably be conducted under constant temperature conditions at the same tissue or body site and on a clean and depilated tissue surface. Likewise influences should be suppressed in which strong ambient light, in particular sun light, could be incident on the measuring zone and distort the measurements in this case.

Exemplary method steps are explained below which are performed in order to determine the unknown tissue parameter from the measured values detected by means of the cited sensor arrangements. In doing so, reference to the determination of blood glucose concentration is made in the following description. It is obvious that virtually any parameter can be taken into account instead of the blood glucose concentration.

The basic idea of the method is to initially determine by means of a self-learning measuring arrangement in an empirical way a correlation between a series of different and basically any arbitrary number of measurement data on the one hand, and the parameter to be measured in the tissue, to initially accumulate a sufficient number of data in this respect, and to ultimately use the ascertained empirical correlation between the measured data and the measured parameter to finally determine the parameter to be determined in an exclusively optical manner. It should be emphasized at this point that the physical correlation which determines the behavior of the irradiated light in the tissue, and the consequently resulting intensity and polarization effects which will then be ultimately measured by the sensor arrangement, need not be known in detail and often can also not be cleared up in detail.

The method is subdivided in two important method stages. In a first method stage, the calibrating phase, a series of so-called measured value vectors are determined and correlated to the parameter determined in another way. In this case, so-called reference vectors are generated. In a second method stage, hereinafter referred to as interpolation phase, the entirety of the measured value and reference vectors determined in the calibrating phase is used to now ascertain the sought tissue parameter from the newly ascertained measured value vectors by way of interpolation.

The dimension of the measured value vectors, i.e. the number of components thereof, as such can be of any size. It is essentially determined by the number of measured values furnished by the sensor arrangements. The sensor arrangement shown in FIG. 2, for example, thus furnishes a first measured intensity value for light scattered on tissue in a first polarization direction, and a second measured intensity value for light scattered in a second polarization direction. Each single measured value vector thus is two-dimensional. A plurality of measured value vectors, together with a tissue parameter respectively allocated to the measured values, hence describes a two-dimensional surface in a three-dimensional space.

In the sensor arrangement of FIG. 8, each single measured value vector consists of four components. The first two components result from the light intensities for the mutually orthogonal polarization directions at the first active laser light source, the third and fourth components of the measured value vector are formed by the polarization-dependent light intensities in case of the second active laser light source. The entirety of the measured value vectors ascertained in this way thus forms a four-dimensional hypersurface in a five-dimensional space.

Accordingly, the measured value vectors ascertained from the sensor arrangement as per FIG. 9 form a five-dimensional hypersurface in a six-dimensional space. If one assumes that in each case the pressure and/or temperature can be added to each sensor arrangement as a further measured value, the dimension of the respective hypersurfaces will increase by one or two.

The method explained below will be presented on the basis of an entirety of two-component measured value vectors. The method steps proceeding in this case, however, may be easily transferred to measured value vectors of higher dimensions, as long as there is only a single tissue parameter to be determined.

The basic idea of the method explained below is to determine firstly the n-dimensional hypersurface of the measured value vectors on the basis of calibrating processes in a sufficiently precise manner, and to subsequently perform interpolations on this hypersurface.

The method starts with a calibrating phase. An exemplary flowchart for this is illustrated in FIG. 11. The sensor arrangement as per FIG. 2 described above is assumed to be used for executing the method. The measured values supplied by sub-array 10 will be subsequently designated by the variable P and an index, the measured values supplied by sub-array 11 by the variable S and an index. The indices designate in this case the number of a respective performed measurement. A measured value vector M hence is composed of the components (P; S). The designation Mᵢ or Mₖ represents in this case a measured value vector of the i-th respectively k-th measurement, the associated components Pᵢ and Si respectively Pₖ and Sₖ are in this case the respective measured values P and S of the iₜₕ respectively kₜₕ measurement. The index i designates in this case measured values and measured value vectors which have been generated during the calibrating phase and for which the tissue parameter had been independently determined, the index k in contrast designates measured value vectors which will be generated during the interpolation phase and for which the tissue parameter is to be interpolated.

In this context, the variable BZ is used hereinafter for the tissue parameter to be determined. The designations BZᵢ respectively BZₖ represent in this case the tissue parameter independently determined in the i-th respectively k-th measurement or interpolated later.

The calibrating phase starts with a method step 41 of independently ascertaining a tissue parameter BZᵢ. Provided that the measurement is a blood glucose measurement, blood will be withdrawn for this purpose and a corresponding blood analysis conducted which delivers an unequivocal measured blood glucose value. At the same time, a non-invasive measurement using the sensor arrangement as per FIG. 2 is performed in a method step 42. The thereby ascertained measured values Sᵢ and Pᵢ constitute a measured value vector Mᵢ and are combined with the independently ascertained tissue parameter BZᵢ to a reference vector Rᵢ and stored in a data base or a memory 44 in a method step 43. The reference vectors stored therein constitute the reference set R of the method.

In a decision step 45, it is checked whether the number of the already detected reference vectors Rᵢ is sufficient. If this is the case, the method proceeds to the interpolation phase 46. The number of reference vectors Rᵢ required for the reference set R depends on the configuration of the hypersurface described by same and the degree of individuality thereof. It has turned out that for blood glucose measurements about 20 reference vectors permit a sufficiently good interpolation later. It applies in general that a number of reference vectors as great as possible of course is advantageous but needs to be reasonably weighted with respect to the justifiable effort.

FIG. 12 shows an exemplary chart for the flow of the interpolation phase 46. The interpolation phase starts with a step 47 in which a measured value vector Mₖ is determined using one of the sensor arrangements cited above. When a sensor arrangement as per FIG. 2 is used, said measured value vector is composed of two components Sₖ and Pₖ. In a next step 48, the reference set R contained in memory 44 is retrieved. The measured value vectors Mᵢ contained in the reference vectors Rᵢ stored therein, are compared with the measured value vector Mₖ in a step 49. In doing so, a predefined number of measured value vectors M′ᵢ, is selected which are closest to the given measured value vector Mₖ. The reference vectors R′ᵢ, assigned to these measured value vectors form the basis for an interpolation step 50 following now. Within interpolation step 50, an interpolated parameter BZₖ is ascertained from the selected reference vectors R′_{I} and the actual measured value vector Mₖ and output as a purely optically and non invasively measured tissue parameter in a step 51.

The described method procedure enables the method to be executed in a self-learning manner. This means that the interpolated parameters BZₖ together with the measured value vectors Mₖ now in turn constitute a reference vector Rᵢ for later measurements. The new reference vectors Rᵢ are added to the data base 44 and the reference set contained in same.

The calculating steps executed in the interpolation phase shall be described hereinafter in more detail. FIG. 13 shows at first an exemplary reference set R from a set of reference vectors R₁ to R₁₀ in the form of a surface embedded in a three-dimensional space. The basis vectors of the three-dimensional space form the parameters P, S and BZ cited above. The reference set thus describes the dependence of the tissue parameter BZ as a function of the measured parameters S and P. Although this function is usually not explicitly known but exists only point by point, it will be assumed for the calculating steps presented below that the surface formed by the reference vectors is fundamentally smooth, i.e. continuous at least at every point.

The reference set R as described is composed of a sufficiently large number of N reference vectors Rᵢ=(Sᵢ, Pᵢ, BZᵢ). If the reference vectors Rᵢ are understood as column vectors of a matrix, the reference set can be indicated as follows:

$$\begin{matrix}R = ( \begin{matrix}{M}_{1} & {M}_{2} & … & {M}_{N} \\ {BZ}_{1} & {BZ}_{2} & … & {BZ}_{N}\end{matrix} ) \\ = ( \begin{matrix}{S}_{1} & {S}_{2} & … & {S}_{N} \\ {P}_{1} & {P}_{2} & … & {P}_{N} \\ {BZ}_{1} & {BZ}_{2} & … & {BZ}_{N}\end{matrix} )\end{matrix} \qquad ( 1 )$$

M₁ to M_{N} constitute in this case the measured value vectors described above.

The distance d between two vectors a=(x₁, y₁) and b=(x₂, y₂) is defined according to the theorem of Pythagoras in the Euclidean space via an establishment of standards:

d=|a−b|=\sqrt{(x₁−x₂)²+(y₁−y₂)²} (2)

Distances dₖᵢ can be determined accordingly to a given measured value vector Mₖ=(Sₖ, Pₖ) and each measured value vector Mᵢ already contained in the reference set as follows:

$$\begin{matrix}{\overline{d}}_{k} = ( \begin{matrix}{d}_{k 1} \\ {d}_{k 2} \\ ⋮ \\ {d}_{ki} \\ ⋮ \\ {d}_{kN}\end{matrix} ) \\ = ( \begin{matrix}\sqrt{{( {S}_{k} - {S}_{1} )}^{2} + {( {P}_{k} - {P}_{1} )}^{2}} \\ \sqrt{{( {S}_{k} - {S}_{2} )}^{2} + {( {P}_{k} - {P}_{2} )}^{2}} \\ ⋮ \\ \sqrt{{( {S}_{k} - {S}_{i} )}^{2} + {( {P}_{k} - {P}_{i} )}^{2}} \\ ⋮ \\ \sqrt{{( {S}_{k} - {S}_{N} )}^{2} + {( {P}_{k} - {P}_{N} )}^{2}}\end{matrix} )\end{matrix} \qquad ( 3 )$$

From this set, the three smallest values d′ₖᵢ, and thus the closest measured value vectors M′ᵢ, and thus the reference vectors R′ᵢ with i=1 . . . 3 required for interpolation are selected from the reference set. This interpolation set I can be indicated in the form of a matrix as follows:

$$\begin{matrix}I = ( \begin{matrix}{M}_{1}^{′} & {M}_{2}^{′} & {M}_{3}^{′} \\ {BZ}_{1} & {BZ}_{2} & {BZ}_{3}\end{matrix} ) \\ = ( \begin{matrix}{S}_{1}^{′} & {S}_{2}^{′} & {S}_{3}^{′} \\ {P}_{1}^{′} & {P}_{2}^{′} & {P}_{3}^{′} \\ {BZ}_{1} & {BZ}_{2} & {BZ}_{3}\end{matrix} )\end{matrix} \qquad ( 4 )$$

These three vectors define a surface in space required for the interpolation. Surfaces can be mathematically defined in a unique manner by a linear combination of the space coordinates x, y and z and a parameter set a′, b′, c′ and d′:

a′x+b′y+c′z=d′. (5)

By introducing new parameters a=−a′/c′, b=−b′/c′ and c=d′/c′, this parameter equation can be transformed into

z=ax+by+C (6)

In this case, z=BZ, x=S, and y=P. Thus

BZ=aS+bP+c. (7)

is valid.

In order to determine the interpolation surface, the parameters A, B and C are thus required to be determined now. For this purpose, use is made of parameter set I, with the result of a linear equation system with three equations and three unknown quantities:

BZ′₁=aS′₁+bP′₁+c

BZ′₂=aS′₂+bP′₂+c

BZ′₃=aS′₃+bP′₃+c (8)

The solutions of this equation system will then result in

$$\begin{matrix}a = \frac{{BZ}_{1}^{′} - {BZ}_{3}^{′} - b ( {P}_{1}^{′} - {P}_{3}^{′} )}{( {S}_{1}^{′} - {S}_{3}^{′} )} & ( 9 ) \\ b = \frac{( {BZ}_{1}^{′} - {BZ}_{2}^{′} ) ( {S}_{1}^{′} - {S}_{3}^{′} ) - ( {BZ}_{1}^{′} - {BZ}_{3}^{′} ) ( {S}_{1}^{′} - {S}_{2}^{′} )}{( {P}_{1}^{′} - {P}_{2}^{′} ) ( {S}_{1}^{′} - {S}_{3}^{′} ) - ( {P}_{1}^{′} - {P}_{3}^{′} ) ( {S}_{1}^{′} - {S}_{2}^{′} )} & ( 10 ) \\ c = {BZ}_{1}^{′} - {aS}_{1}^{′} - {bP}_{1}^{′} & ( 11 )\end{matrix}$$

Thus, the interpolated value for the biological tissue parameter BZₖ assigned to the measured value vector Mₖ will result from the relation:

BZₖ=aSₖ+bPₖ+c (12)

A value of zero occasionally arising in the denominator of equations (9) or (10) can be removed by mutually exchanging, i.e. permuting columns from equation (3).

Reference should be made to FIGS. 13 and 14 for illustrating the stated interpolation steps. FIG. 13 shows a section of a reference set formed by the end points of reference vectors R₁ to R₁₀ in a three-dimensional (S; P; BZ) space. The reference set is in this case a two-dimensional hypersurface. FIG. 14 shows a measured value vector Mₖ with an associated interpolated tissue parameter BZₖ in the environment of the three closest reference vectors R′₁ to R′₃. Same constitute the interpolation set I selected in this case. Same form an interpolation surface F. As can be seen from the figure, the interpolated parameter BZₖ can be understood as the value allocated to the measured value vector Mₖ on the area of the interpolation surface F.

Two things can be read in the representation of FIG. 14. Firstly, the interpolation becomes then particularly precise when the hypersurface is as flat and free from curves as possible, and its precision even increases when measured value vectors of the reference set are as close as possible to the measured value vector whose parameter BZₖ needs to be interpolated. Secondly, the reference vectors constitute invariable points of support for the otherwise unknown hypersurface. Same is approximated in each new interpolation procedure by a new interpolation surface in a small area, with the interpolated tissue parameter being slightly above or below the real hypersurface. This is of importance for the subsequent interpolation procedures in which use is made again of interpolated tissue parameters BZₖ and associated measured value vectors Mₖ. Strictly speaking, it is no longer the question of an interpolation along a clearly defined surface, but one within a point cloud more or less restricted to a certain area. The basic method steps are thereby not changed, but it is evident that the interpolation will be all the more precise, the more marked and distinct the optically determined measured parameters depend on the tissue parameter BZ to be determined.

Alternatively, the interpolation can also be performed by means of an interpolation mesh created from the reference set R. In this case, the value range of the values of measured values S and P is subdivided into a mesh of 12 by 12 points, for instance, and the reference vectors Rᵢ, i.e. the reference values BZᵢ are ascertained at these points in a first interpolation step. The interpolation mesh allows reference measured value vectors Mᵢ situated close to each measured value vector Mₖ to be identified in the interpolation phase and thus the interpolation to be performed in a more secure manner.

The reference set, i.e. the hypersurface formed by the reference vectors can exhibit a quite complex shape. FIG. 15 shows to that end an example obtained from real calibration measurements. In the diagram, blood glucose concentrations BZ are plotted versus the measured values S and P in arbitrary units. The reference set presents itself in this example as a surface formed by maxima, minima and saddle points, which can be quite different from tissue to tissue or test person to test person and thus can also be rated for the test person or examined tissue as being an individual “fingerprint”.

On the operating side, these evaluation procedures are performed as background processes of a user-friendly menu navigation. This menu navigation is of particular advantage in collecting measurement series intended to be performed personalized to one test person. In this case, the user can firstly select and confirm a test person's name from a first menu. During the calibrating phase, a measurement is performed and the user thereupon directly requested to enter the independently determined value BZᵢ for the tissue parameter. The entries are confirmed by the device and stored within a personalized data base. The input of the respective numeric values for BZᵢ can in this case be performed via a number keypad or via UP and DOWN menus in which the respective values are scrolled through within a sufficiently sized selection area.

In doing so, is also possible to browse within already existing reference data and to edit or else cancel this data. This browse function can be performed both on the device itself and on an external data processing unit via the mentioned interface and using the more extensive and convenient editing options there, e.g. corresponding evaluation programs and text editors.

When a certain amount of reference data is reached, the device will output a corresponding indication via the display and signalize therewith that the interpolation phase can be started. During the interpolation phase, the measurement is performed just as in the calibrating phase. After performing the measurement, the device will, however, not output a request for entering a reference value, rather it displays the execution of the interpolation procedure described above on the display screen. The tissue parameter BZₖ interpolated on this occasion is displayed and internally stored. Also in this case it is possible to transmit the data captured in the measuring process via the interface to the external data processing unit and to perform further processing activities there.

It is basically possible to modify boundary conditions and to indicate under which criteria an interpolation should be performed, and under which criteria the interpolation should be omitted. For this purpose, the user can specify via the menu, for instance, certain maximum amounts for the distances dₖᵢ cited above. If the distance dₖᵢ between the measured value vector Mₖ and the measured value vector Mᵢ of the reference set is outside this predefined range, a corresponding indication will be output and the interpolation stopped or continued with the proviso that a determination of the tissue parameter would possibly be highly erroneous.

A software component contained in the external data processing unit corresponds to the software contained in the device. Same consist of a set of program tools for data analysis. It permits the hypersurface generated from the measured value vectors and tissue parameters to be represented and thus the quality of an optional interpolation to be judged.

The software moreover comprises components for comparing the correct and independently determined tissue parameters BZᵢ with the calculated values BZₖ on the basis of the optical measurements and displays the quality of the optical measurements in a graph. Thus, an optional additional quality check of the measurement is made possible.

The software moreover comprises means for calculating a correlation function between the independently determined tissue parameters BZᵢ and the interpolated values BZₖ.

In order to execute these program tools, the respective measurement data is transferred to the external data processing unit. After executing the program means, a file including the respective results is generated and output. For performing these procedures, for example, use is made of a combination of data processing software and already specified means for representing data and the output thereof. The measured values, for instance, are present in the form of a file in ASCII format and are subjected to a corresponding data analysis by a first software means. The results calculated on this occasion are transferred into a file which in turn is accessed by a plot program, e.g. gnuplot. The data calculated in this case, in particular the hypersurface for the interpolation or the correlation function is now represented by means of gnuplot and subsequently converted into a LaTeX-compatible file format. Finally, the LaTeX file is completed by corresponding text information and transferred into a DVI, PS or PDF file by means of a compiling program and displayed. As an alternative to this, the respective values can even be transferred into a graphical display program and viewed on a display.

The associated code comprises five sections, for instance. In a first section, the variables necessary for executing the program are defined. In a second section, configuration data are read in. The data is subsequently read out from a data file in a third section, and the computed data are written into an output file in a fourth section. The fifth section constitutes the actual core of the code and is designed to compute the correlation values.

Use is appropriately made of the configuration file already stored in advance for reading in configuration data. The program then outputs the read data files as information and defines the file names for the output values. The storage space for the output data is thus reserved.

In a next step, the input file is checked for its proper format. Subsequently, the percentage difference x between the correct value BZᵢ and the value BZₖ is ascertained for each value BZ:

$$x = 100 \frac{{BZ}_{k}}{{BZ}_{i}} - 100 \qquad ( 13 )$$

For calculating the correlation function, for example, use can be made of Pearson's Product Moment respectively Pearson Coefficient. Same is a dimensionless measure for the degree of a linear correlation between two at least interval-scaled features. It can adopt values between −1 and +1. In case of a value of +1 or −1, there is a completely positive (or negative) linear correlation between the considered features. If the correlation coefficient has the value 0, there is no linear dependence at all between the two features. The Pearson Coefficient is calculated for N values BZᵢ and N values BZₖ as follows:

$${ρ}_{ik} = \frac{{\sum }_{n = 1}^{N} ( {BZ}_{in} \cdot {BZ}_{kn} ) - \frac{1}{n} {\sum }_{n = 1}^{N} {BZ}_{in} {\sum }_{n = 1}^{N} {BZ}_{kn}}{\sqrt{{\sum }_{n = 1}^{N} {BZ}_{in}^{2} - \frac{1}{N} {( {\sum }_{n = 1}^{N} {BZ}_{in}^{2} )}^{2} \cdot } \sqrt{{\sum }_{n = 1}^{N} {BZ}_{kn}^{2} - \frac{1}{N} {( {\sum }_{n = 1}^{N} {BZ}_{kn}^{2} )}^{2}}} \qquad ( 14 )$$

The device according to the invention and the method according to the invention have been described in more detail on the basis of exemplary embodiments. Further possible embodiments will be obvious to the person skilled in the art. Same will result in particular from the dependent claims.

### LIST OF REFERENCE NUMERALS

1 central unit
1a energy supply unit
2 laser operating unit
3 laser source
4 sensor unit
4a measuring sensor
5 control unit
5a additional sensor
6 storing and processing unit
7 interface
8 external data processing unit
9 sensor array
10 first sub-array
11 second sub-array
12 first polarizer
13 second polarizer
14 light outlet opening
15 spacer
16 tissue surface
17 pressure sensor
18 temperature sensor
19 slot
20 fiber-optic light cable
21 tilting mechanism
22 measuring sensor, absolute measurement
23 deflecting mirror
24 measuring sensor, scatter measurement
25 first laser light source
26 second laser light source
27 annular detector
28 photo detector, absorption measurement
29 photo detector, refraction measurement
30 photo sensor, spectrally resolving
31 photo sensor for polarization state
32 laser source
33 inner light source
34 first reflecting surface
35 second reflecting surface
36 opening
37 polarization foil
38 first annular detector
39 second annular detector
41 independently ascertaining a tissue parameter
42 non-invasive optical measurement
43 generating a reference vector
44 storing in memory
45 integrity check
46 transition to interpolation phase
47 determining a measured value vector
48 calling up a reference set
49 comparing measured value vector/reference vector
50 interpolation
51 outputting an interpolated tissue parameter

1. A device for determining a biological, chemical and/or physical parameter in living biological tissue comprising
a central unit (1), an energy supply unit (1a), a laser operating unit (2) for operating at least one laser source (3) directed onto the biological tissue, at least one sensor unit (4) for detecting light scattered back and/or absorbed by the biological tissue, a control unit (5), a storing and processing unit (6), and an interface (7) for an external data processing unit (8).

2. The device according to claim 1,
characterized in that
the sensor unit (4) includes a planar sensor array (9), wherein the sensor array comprises a first sensor portion as an inner sub-array (10), and a second sensor portion as an outer sub-array (11) surrounding the inner sub-array.

3. The device according to claim 1,
characterized in that
the inner sub-array (10) comprises an attachment having a first polarizer (12) oriented in a first polarization direction, and the outer sub-array (11) comprises an attachment having a second polarizer (13) oriented in a second polarization direction, wherein the polarization direction of the first polarizer is oriented perpendicular to the polarization direction of the second polarizer.

4. The device according to claim 1,
characterized in that
the sensor unit (4) comprises a photometer unit comprising a first photometer (22) for determining an absolute intensity of the light from the laser source (3), and a second photometer (24) for measuring the light scattered by the tissue.

5. The device according to claim 4,
characterized in that
the sensor unit (4) comprises a change-over mechanism (23) for redirecting the light from the laser source to the first photometer (22) as required.

6. The device according to claim 1,
characterized in that
two laser sources (25, 26) having mutually orthogonal beam directions are provided.

7. The device according to claim 1,
characterized in that
the laser source (3, 25, 26) comprises an exit opening arranged in the sensor unit, wherein the exit opening has a beam direction inclined at a tilt angle (α) with respect to the detection direction of the sensor array.

8. The device according to claim 7,
characterized in that
the tilt angle (α) has a value adjustable to about 45°.

9. The device according to claim 1,
characterized in that
the first sub-array (10) consists of at least one first single diode and the second sub-array (11) of at least four single diodes which are uniformly distributed around the first single diode.

10. The device according to claim 1,
characterized in that
the sensor unit comprises at least one pressure sensor (17) for measuring a contact pressure between the sensor unit and the tissue, and/or at least one temperature sensor (18) for measuring a tissue temperature.

11. The device according to claim 1,
characterized in that
the laser operating unit (2) comprises a pulse unit for generating laser pulses, and a program unit for programming and executing laser pulse sequences and/or changing light intensity.

12. The device according to claim 1,
characterized in that
the pressure sensor (17) and/or the temperature sensor (18) form a control loop cooperating with the control unit (5) for adjusting an appropriate contact pressure and/or an appropriate temperature value.

13. A method for determining a biological, chemical and/or physical parameter (BZ) in a living biological tissue in the form of a self-learning process flow including the following process steps:
realizing a calibrating phase comprising at least one conventional determination of the parameter in conjunction with at least one light scatter measurement performed on the tissue for ascertaining optical measured values,
assigning the at least one conventionally determined parameter to the respective optical measured values, and storing a calibrating reference set,
realizing an interpolation phase comprising at least one light scatter measurement performed on the tissue for ascertaining optical measured values,
interpolating the parameter from the measured values of the light scatter measurement and the data of the reference set, and storing the interpolated parameter in the reference set.

14. The method according to claim 13,
characterized in that
when realizing the calibrating phase, the ascertaining of a reference set (R) is performed in the form of reference vectors (Rᵢ), wherein each reference vector consists of the conventionally ascertained parameter (BZᵢ) and a measured value vector (Mᵢ) containing the optical measured values,
when realizing the interpolation phase, a measured value vector (Mₖ) is determined with optical measured values, and the associated interpolated parameter (BZₖ) together with the measured value vector (Mₖ) is transferred into the reference set as a new reference vector (Rₖ).

15. The method according to claim 13,
characterized in that
the measured value vector (Mᵢ) ascertained when realizing the calibrating phase includes a light intensity influenced by the tissue in a first polarization direction (Sᵢ), and a light intensity influenced by the tissue in a second polarization direction (Pᵢ), and the measured value vector (Mᵢ) is combined with the independently ascertained parameter (BZᵢ) to result the reference vector (Rᵢ).

16. The method according to claim 13,
characterized in that
the measured value vector (Mₖ) ascertained when realizing the interpolation phase includes a light intensity influenced by the tissue in a first polarization direction (Sₖ), and a light intensity influenced by the tissue in a second polarization direction (Pₖ).

17. The method according to claim 13,
characterized in that
the interpolated parameter (BZₖ) is ascertained using the following steps:
registering the measured value vector (Mₖ) and ascertaining closest measured value vectors (M′ᵢ) from the reference set (R) having a minimum distance to the measured value vector (Mₖ),
interpolating the parameter (BZₖ) assigned to the measured value vector (Mₖ) from the closest measured value vectors (M′i) and the respectively associated reference parameters (BZᵢ).

18. The method according to claim 13,
characterized in that
the interpolated parameter (BZₖ) is added to the reference set (R) together with the measured value vector (Mₖ) after realizing the interpolation.
